(19) United States
(12) Patent Application Publication
(10) Pub. No.: US 2010/0036095 A1
(43) Pub. Date: Feb. 11, 2010

(54) Human CD59 mutants with modulated complement binding activity

(76) Inventor: Stephen Tomlinson, Mt. Pleasant, SC (US)

Correspondence Address: Ballard Spahr LLP, SUITE 1000, 999 PEACHTREE STREET, ATLANTA, GA, 30309-3915, US

(21) Appl. No.: 11/997,650

(22) PCT Filed: Aug. 1, 2006
(86) PCT No.: PCT/US06/29958

§ 371 Date: Aug. 11, 2008

Related U.S. Application Data
(60) Provisional application No. 60/704,356, filed on Aug. 1, 2005.

(51) Int. Cl. C07K 14/705

(52) U.S. Cl. 530/350

(57) ABSTRACT

Disclosed are compositions and methods using mutant CD59. Also disclosed is new insight into the CD59-C8/C9 binding interface and engineered soluble CD59 molecules with significantly improved complement inhibitory activity.

This application claims the benefit of U.S. Provisional Application No. 60/704,356 filed on Aug. 1, 2005, which application is incorporated herein by reference in its entirety.

This invention was made with government support under grant to the United States National Institutes of Health R01 AI047386. The government has certain rights in the invention.

### I. BACKGROUND

Complement is an important component of host defense and is an effector mechanism for both innate and adaptive immune responses. Complement also plays important roles in enhancing the induction of both humoral and cellular immunity, regulating tolerance to self-antigens, and in the clearance of immune complexes and apoptotic cells. These effects of complement are mediated either directly or indirectly by bioactive cleaved protein fragments, or by a terminal cytolytic protein assembly, termed the membrane attack complex (MAC or C5b-9). Generation of the MAC during the complement cascade is initiated by cleavage of C5, which yields C5b and results in the sequential binding of C6, C7, C8 and multiple C9 molecules. Necessarily, complement effector mechanisms are under tight control to prevent damage to host cells, and MAC formation is under the control of CD59, a widely distributed 18-21-kD (77 amino acids) glycoprotein attached to the plasma membrane by a glycosyl-phosphatidylinositol (GPI) anchor. CD59 functions by binding to C8 and C9 in the assembling MAC and interfering with membrane insertion and pore formation.

Under certain disease conditions, such as autoimmune disease and inflammatory conditions, inappropriate or excessive complement activation occurs and complement control mechanisms, including CD59 function, are broken down or overcome. The MAC has been implicated as a key player in causing tissue injury in many of these pathological states (reviewed in (Arumugam, T. V., et al. (2004)Shock 21, 401-409, Morgan, B. P., and Harris, C. L. (2003) Mol Immunol 40(2-4), 159-170, Sahu, A., and Lambris, J. D. (2000) Immunopharmacology 49(1-2), 133-148, Quigg, R. J. (2002) Trends Mol Med 8, 430-436, Lambris, J. D., and Holers, V. M. (eds). (2000) Therapeutic interventions in the complement system, Humana Press, Totowa, N.J.). From a clinical standpoint, understanding the molecular interaction between CD59 and its complement ligands may assist with the design and engineering of effective recombinant soluble CD59-based therapeutics to limit MAC-dependent disease pathology. CD59 expression has also been implicated in tumorigenesis and in providing cancer cells with protection from monoclonal antibody immunotherapy (Chen, S., et al. (2000) Cancer Res. 60, 3013-3118, Shapiro, A., et al. (1984) Cancer Res 44(7), 3051-3054, Gelderman, K. A., et al. (2004) Trends Immunol 25(3), 158-164), and has been shown to be upregulated in some cancers indicating a role in immune resistance (Fishelson, Z., et al. (2003) Mol Immunol 40(2-4), 109-123, Murray, K. P., et al. (2000) Gynecol Oncol 76(2), 176-182, Thorsteinsson, L., et al. (1998) APMIS 106(9), 869-878, Xu, C., et al. (2005) Prostate 62(3), 224-232).

### II. SUMMARY

Disclosed are methods and compositions related to modified CD59 molecules. Also disclosed herein are methods of treating conditions such as inflammatory conditions, viral infection, bacterial infections, parasitic infections, fungal infections, and cancers using the disclosed modified CD59 molecules.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effect of alanine substitutions on CD59 complement inhibitory activity. CD59 activity was determined by assaying complement sensitivity of CHO cells expressing similar levels of wt or mutant CD59 (* p<0.05, ** p<0.01, *** p<0.001). Mean+/−SD, n=5-9

FIG. 2 shows the putative C8/C9 binding site and single alanine mutation data. Side chains of residues potentially involved in binding are presented with a skin representation and colored by relative inhibitory activity. N48 and T52 showed observable but insignificant changes in activity. (p>0.05) K41, E43, and D49 did not show any change and are presented with a stick representation.

FIG. 3 shows the effect of various substitutions on CD59 complement inhibitory activity. 1: 29A21A, 2: 29A23A, 3: 51A20A, 4: 51A23A, 5: 51A29A, 6: 20A23A, 7: 20-23A, 8: 20-23G, 9: 51A plus 20-23A, 10: 23G, 11: 29A18Q, 12: 5R, 13: 7R, 14: 9R (* p<0.05, ** p<0.01, ***p<0.001). Mean+/−SD, n=5-9.

FIG. 4 shows the inhibition of complement mediated lysis by wild type and mutant CR2-CD59 proteins. CHO cells were sensitized with antibody and incubated with normal human serum containing CR2-CD59 protein. Mean+/−SD, n=5.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Treatment” or “treating” means to administer a composition to a subject with a condition, wherein the condition can be any pathogenic disease, autoimmune disease, cancer or inflammatory condition. The effect of the administration of the composition to the subject can have the effect of, but is not limited to, reducing the symptoms of the condition, a reduction in the severity of the condition, or the complete ablation of the condition.

Herein, “inhibition” or “inhibits” means to reduce activity. It is understood that inhibition can mean a slight reduction in activity to the complete ablation of all activity. An “inhibitor” can be anything that reduces activity.

Herein, “activation” or “activates” means to increase activity. It is understood that activation can mean an increase in existing activity as well as the induction of new activity. An “activator” can be anything that increases activity.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

the disclosed compositions relate to modified cd59 molecules and methods of using the modified CD59 molecules to treat conditions.

The determination of CD59 3D structure by NMR revealed a single cysteine rich domain composed of two β-sheets running anti-parallel to each other and a short helix (Kieffer, B., et al. (1994) Biochemistry 33(15), 4471-4482, Fletcher, C. M., et al. (1994) Structure 2(3), 185-199. Previous studies of the CD59 binding interface indicate that its C8 (C5b-8) and/or C9 (C5b-9) binding site is located in the vicinity of a hydrophobic groove on the membrane distal face of the protein centered around residue W40 and close to the helix (Yu, J., et al. (1997) Journal of Experimental Medicine 185, 745-753, Bodian, D. L., et al. (1997) Journal of Experimental Medicine 185, 507-516). Mutational strategies used to putatively identify the CD59 binding interface have been based on either the rational selection of single residues or the production of chimeric proteins containing functional and nonfunctional domains. The selection of residues for site-specific mutation have primarily been based on their predicted positions at the protein surface, or the identification of evolutionarily conserved residues (Yu, J., et al. (1997) Journal of Experimental Medicine 185, 745-753, Bodian, D. L., et al. (1997) Journal of Experimental Medicine 185, 507-516, Petranka, J., et al. (1996) Blood Cell. Mol. Dis. 22, 281-295, Hinchliffe, S. J., and Morgan, B. P. (2000) Biochemistry 39(19), 5831-5837, Zhang, H.-F., et al. (1999) J. Biol. Chem. 274, 10969-10974, Zhao, X. J., et al. (1998) J. Biol. Chem. 273, 10665-10671). The chimeric approach has involved swapping CD59 domains from different species that are known to function in a species selective manner (Huesler, T., et al. (1995) J. Biol. Chem. 270, 3483-3486, Yu, J., et al. (1997) Biochem. 36, 9423-9428), or by swapping domains between human CD59 and mouse Ly6E, a structural, but not functional analog of CD59 (Yu, J., et al. (1997) Journal of Experimental Medicine 185, 745-753).

The only full-atom structural data currently available for CD59 comes from several NMR models (PDB 1cdq, 1cdr, 1cds, 1erg) (Kieffer, B., et al. (1994) Biochemistry 33(15), 4471-4482, Fletcher, C. M., et al. (1994) Structure 2(3), 185-199. However, those models have a number of potential defects, including a deformed alpha helix (F47-E56) and partial separation of the C6-C13 loop from the rest of the structure, producing a continuous channel through the protein. Structure 1erh does not share the channel but is missing residues F71-N77. Additionally upon visual inspection, the current models show side chain packing significantly less compact than would be expected of typical high-resolution crystallographic structures. Such defects make interpreting mutational data from a structural standpoint difficult, as one cannot be sure of the spatial relationships between side chains. The ability to perform energy-based geometrical analysis or docking is similarly compromised.

Disclosed herein are mutant CD59 molecules (mutCD59), and fragments thereof that have been modified from the native structure by substituting one or more amino acids. It is understood and herein contemplated that the term “mutCD59” and “modified CD59” can refer to the same molecule and can be used interchangeably throughout the application. It is also understood that a “native structure” refers both the sequence and folding confirmation of a protein without substitutions. Thus, for example, the nucleotide and amino acid sequences of CD59, represented by SEQ ID NO:1 and 2 respectively, are the native structure of human CD59. It is understood and contemplated herein that a substitution of a single nucleotide can change the amino acid residue at a particular site and this substitution can affect the folding pattern of the protein. In particular, such substitutions can affect the binding groove of CD59 allowing greater access to the binding site or reducing access to the binding site. Without being bound by theory, it is contemplated that by modulating the access to the binding site of CD59 by opening or closing the binding groove, the activity of CD59 can be modulated. For example, a substitution that opens the binding groove thus making the binding site of CD59 more accessible will increase the inhibitory activity of CD59. A substitution that closes the binding groove thus making the binding site of CD59 more accessible will decrease the inhibitory activity of CD59.

The substitutions disclosed herein can comprise any change in the native structure that modulates CD59 inhibitory activity, and in particular, those substitutions that open the binding groove of CD59 allowing greater access into the binding site of CD59. Thus, for example, a substitution can comprise the removal of a polar (Serine, Threonine, Methionine, Asparagine, and Glutamine), aromatic (Phenylalanine, Tyrosine, and Tryptophan), or charged (Lysine, Arginine, Histidine, Aspartate, and Glutamate) amino acid residue for substitution with a nonpolar residue (Alanine, Glycine, Valine, Leucine, Isoleucine, and Proline). Alternatively, the substitution may comprise the substitution of an amino acid with a bulky side chain with an amino acid with a small side chain. Thus, for example, a substitution may comprise the substitution of a polar, aromatic, or charged residue for Alanine (e.g., substituting Phenylalanine with Alanine at position 23 (F23A)). It is also contemplated herein that the substitution does not occur at a position where the native amino acid residue is a Cysteine as such substitutions can disrupt disulfide bonds that are important to global protein folding. It is further contemplated that the particular amino acids that will affect the binding groove of CD59 are between residues 16 and 57 of the 77 amino acid protein human CD59. Thus, specifically disclosed herein are modified CD59 molecules wherein the molecule has a first amino acid substitution between residues 16 and 57, wherein the substitution modulates the inhibitory activity of CD59, wherein the substitution does not change a cysteine, and wherein the substitution is not at residue 40.

The disclosed mutCD59 molecules can comprise a substitution at any residue along the face of the binding groove or immediately adjacent to the binding groove of CD59, wherein the substitution modulates the inhibitory activity of CD59. Thus, for example, the disclosed modified CD59 molecules can comprise a substitution along the exposed face of the binding groove at residues S20, D22, F23, L27, T29, L33, Q34, Y36, N37, K38, W40, F42, K41, R53, L54, or R55. Also contemplated, for example, are mutCD59 molecules comprising a substitution immediately adjacent to the exposed face of the binding groove at residues S21, T51 or N57. As disclosed, it is contemplated herein that the disclosed modified CD59 molecules can comprise an amino acid substitution at residues on the exposed face of the groove or adjacent to the groove. It is also contemplated that the substitutions can comprise the substitution of the native residue at a particular position with alanine. Therefore, disclosed herein are modified CD59 molecules of the invention wherein the substitution can be selected from the group consisting of S20A, S21A, D22A, F23A, L27A, T29A, L33A, Q34A, Y36A, N37A, K38A, W40A, F42A, K41A, T51A, R53A, L54A, R55A, and N57A. It is understood and herein contemplated that the amino acid references to a particular residue or alternatively an amino acid at position X refer to the native CD59 amino acid sequence (SEQ ID NO:2) (Davies et al. (1989) J. Exp. Med. 170 (3), 637-654; incorporated herein by reference in its entirety for its teachings of CD59). Thus, for example, CD59(T51A) refers to a mutCD59 wherein the native threonine at position 51 is substituted with an alanine.

The disclosed compositions can further comprise a targeting moiety to direct the CD59, mutCD59, or fragment thereof to complement activity (e.g., CR2). Thus specifically contemplated and disclosed herein are compositions comprising CD59, mutCD59, or fragments thereof, further comprising CR2. For example, the disclosed CD59, mutCD59, or fragment thereof can be synthesized as a fusion protein or immunoconjugate with CR2.

CR2 consists of an extracellular portion consisting of 15 or 16 repeating units known as short consensus repeats (SCRs). Amino acids 1-20 comprise the leader peptide, amino acids 23-82 comprise SCR1, amino acids 91-146 comprise SCR2, amino acids 154-210 comprise SCR3, amino acids 215-271 comprise SCR4. The active site (C3dg binding site) is located in SCR 1-2 (the first 2 N-terminal SCRs). SCR units are separated by short sequences of variable length that serve as spacers. It is understood that any number of SCRs containing the active site can be used. In one embodiment, the construct contains the 4 N-terminal SCR units. In another embodiment, the construct includes the first two N-terminal SCRs. In another embodiment the construct includes the first three N-terminal SCRs.

It is understood and herein contemplated that other targeting moitiess can be used in conjunction with the CD59 and mutCD59 of the invention to target to sites inflammation. For example, K9/9 and P-selectin glycoprotein ligand, as well as, antigen targeting antibodies such as an anti-gp120 antibody (for HIV treatment) can be used.

Herein a “fusion protein” means two or more components comprising peptides, polypeptides, or proteins operably linked. CD59 and mutants thereof can be linked to complement targeting moieties by an amino acid linking sequence. Examples of linkers are well known in the art. Examples of linkers can include but are not limited to (Gly₄Ser)₂, (Gly₄Ser)₃ (G4S), (Gly₃Ser)₄ (G3S), SerGly₄, and SerGly₄SerGly₄. Linking sequences can also consist of “natural” linking sequences found between SCR units within human (or mouse) proteins, for example VSVFPLE, the linking sequence between SCR 2 and 3 of human CR2, can be used to link the mutant CD59s of the invention with CR2. Fusion proteins can also be constructed without linking sequences.

Also disclosed are compositions, wherein the construct is an immunoconjugate. Herein “immunoconjugate” means two or more components comprising peptides, polypeptides, or proteins operably linked by a chemical cross-linker. Linking of the components of the immunoconjugate can occur on reactive groups located on the component. Reactive groups that can be targeted using a cross-linker include primary amines, sulfhydryls, carbonyls, carbohydrates and carboxylic acids, or active groups can be added to proteins. Examples of chemical linkers are well known in the art and can include but are not limited to bismaleimidohexane, m-maleimidobenzoyl-N-hydroxysuccinimide ester, NHS-Esters-Maleimide Crosslinkers such as MBS, Sulfo-MBS, SMPB, Sulfo-SMPB, GMBS, Sulfo-GMBS, EMCS, Sulfo-EMCS; Imidoester Cross-linkers such as DMA, DMP, DMS, DTBP; EDC [1-Ethyl-3-(3-Dimethylaminopropyl)carbodiimide Hydrochloride], [2-(4-Hydroxyphenyl)ethyl]-4-N-maleimidomethyl)-cyclohexane-1-carboxamide, DTME: Dithio-bis-maleimidoethane, DMA (Dimethyl adipimidate.2 HCl), DMP (Dimethyl pimelimidate.2 HCl), DMS (Dimethyl suberimidate.2 HCl), DTBP (Dimethyl 3,3′-dithiobispropionimidate.2 HCl), MBS, (m-Maleimidobenzoyl-N-hydroxysuccinimide ester), Sulfo-MBS (m-Maleimidobenzoyl-N-hydroxysuccinimide ester), Sulfo-SMPB (Sulfosuccinimidyl 4-[p-maleimidophenyl]butyrate(, GMBS (N-[.-maleimidobutyryloxy]succinimide ester), EMCS-N-[.-maleimidocaproyloxy]succinimide ester), and Sulfo-EMCS(N-[.-maleimidocaproyloxy]sulfosuccinimide ester).

Disclosed are methods of treating a condition affected by complement in a subject comprising administering to the subject the composition of the invention. It is understood that administration of the composition to the subject can have the effect of, but is not limited to, reducing the symptoms of the condition, a reduction in the severity of the condition, or the complete ablation of the condition.

1. Homology/Identity

It is understood that one way to define any known variants and derivatives or those that might arise, of the disclosed genes and proteins herein is through defining the variants and derivatives in terms of homology to specific known sequences. For example SEQ ID NO: 1 sets forth a particular nucleotide sequence of a CD59 and SEQ ID NO: 2 sets forth a particular amino acid sequence of the protein encoded by SEQ ID NO: 1, a CD59 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. More particularly, the variants can have at least 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Peptides

a) Protein Variants

As discussed herein there are numerous variants of the CD59 protein and mutCD59 protein that are known and herein contemplated. In addition, to the known functional CD59 or mutCD59 strain variants there are derivatives of the CD59 or mutCD59 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative
Substitutions, others are known in the art.

Alaser
Arglys, gln
Asngln; his
Aspglu
Cysser
Glnasn, lys
Gluasp
Glypro
Hisasn; gln
Ileleu; val
Leuile; val
Lysarg; gln;
MetLeu; ile
Phemet; leu; tyr
Serthr
Thrser
Trptyr
Tyrtrp; phe
Valile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:1 sets forth a particular nucleotide sequence of CD59 and SEQ ID NO:2 sets forth a particular amino acid sequence of a CD59 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989; Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:2 is set forth in SEQ ID NO:1 In addition, for example, a disclosed conservative derivative of SEQ ID NO:2 is shown in SEQ ID NO: 8, where the isoleucine (I) at position 3 is changed to a valine (V). It is understood that for this mutation all of the nucleic acid sequences that encode this particular derivative of the CD59 are also disclosed. It is also understood that while no amino acid sequence indicates what particular DNA sequence encodes that protein within an organism, where particular variants of a disclosed protein are disclosed herein, the known nucleic acid sequence that encodes that protein in the particular CD59 or mutCD59 from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### C. COMPLEMENT MODULATION

It is understood and herein contemplated that the disclosed CD59 and mutCD59 proteins can be used to modulate complement activity through their effect on membrane attack complex (MAC) formation. Therefore, disclosed herein are compositions comprising CD59, mutCD59, or fragments thereof, wherein the substitution modulates the inhibitory activity of CD59. It is contemplated herein that the inhibitory activity of CD59 can increase or decrease due to the substitution. Therefore, disclosed herein are methods of modulating membrane attack complex (MAC) formation in a subject comprising administering to the subject the modified CD59 of the invention.

### D. METHODS OF USING THE COMPOSITIONS TO INHIBIT COMPLEMENT

Disclosed herein are modified CD59 molecules wherein the substitution increases the inhibitory activity of CD59. It is understood that increasing the inhibitory activity of CD59 decreases MAC formation and thus decreases complement dependent pathology. Thus, for example, provided are modified CD59 molecules of the invention wherein the modified CD59 molecule increases the inhibitory activity of CD59 and wherein the modified CD59 molecule comprises a substituted residue selected from the group of residues consisting of 20, 21, 22, 23, 27, 29, 37, 51, 53, 54, and 57.

It is also contemplated herein that the disclosed modified CD59 molecules can comprise more than one substitution. Thus, disclosed herein are mutCD59 molecules comprising a substitution at 1, 2, 3, 4, 5, 6, 7, 8, 9, 10, 11, 12, 13, 14, or 15 amino acid residues, wherein the substituted residues can be selected from the group consisting of N18Q, S20A, S21A, D22A, F23A, F23G, L27A, T29A, L33A, Q34A, Y36A, N37A, K38A, W40A, F42A, K41A, T51A, R53A, L54A, R55A, and N57A. It is understood and herein contemplated that multiple substitutions in the modified CD59 molecules of the invention can have an additive effect on the increase of the inhibitory activity of CD59 relative to a single substitution. Thus, for example, disclosed herein are modified CD59 molecules of the invention wherein the modified CD59 further comprises a second substitution at residues 20, 21, 22, 23, 27, 29, 37, 48, 51, 53, 54, or 57. Thus, for example, disclosed herein are modified CD59 molecules of the invention comprising substitutions at residues 20 and 51, 21 and 29, 23 and 29, 23 and 51, and 29 and 51. The substitutions at these residues can be alanine.

Disclosed are methods of treating a condition comprising administering to a subject with a condition, the modified CD59 fusion protein of the invention, wherein the modified CD59 molecule increases CD59 inhibition of MAC, and wherein the inhibition of MAC decreases MAC dependent disease pathology. Thus, one effect a increasing complement inhibition is decreasing the effects of complement. It is understood that the effect of the administration of the composition to the subject can have the effect of but is not limited to reducing the symptoms of the condition, a reduction in the severity of the condition, or the complete ablation of the condition.

Disclosed are methods of the invention, wherein the condition treated is an inflammatory condition. Also disclosed are methods of the invention, wherein the inflammatory condition can be selected from the group consisting of asthma, systemic lupus erythematosus, rheumatoid arthritis, reactive arthritis, spondylarthritis, systemic vasculitis, insulin dependent diabetes mellitus, nephritis, Proteinuria, Diabetic nephropathy, Focal and segmental glomerulosclerosis, Membranous nephropathy, IgA nephropathy, Lupus nephritis, Minimal change disease, Amyloidosis, Membranoproliferative glomerulonephritis, Essential mixed cryoglobulinemia (includes secondary to hepatitis C), Light chain deposition disease, Vasculitis (includes Wegener's granulomatosis, microscopic polyangiitis and renal limited vasculitis), Congenital nephrotic syndrome, Fibrillary glomerulonephritis, Mesangial proliferative, glomerulonephritis, Postinfectious glomerulonephritis, Drug-induced nephrotic syndrome, Preeclampsia/eclampsia, Hypertensive nephrosclerosis, Immunotactoid glomerulonephritis, multiple sclerosis, experimental allergic encephalomyelitis, Sjögren's syndrome, graft versus host disease, inflammatory bowel disease including Crohn's disease, ulcerative colitis, ischemia reperfusion injury, myocardial infarction, alzheimer's disease, acute and chronic transplant rejection (allogeneic and xenogeneic), thermal trauma, traumatic injury, any immune complex-induced inflammation, myasthenia gravis, cerebral lupus, Guillain-Barre syndrome, vasculitis, systemic sclerosis, anaphlaxis, catheter reactions, atheroma, infertility, thyroiditis, ARDS, post-bypass syndrome, hemodialysis, juvenile rheumatoid, Behcets syndrome, hemolytic anemia, pemphigus, bullous pemphigoid, stroke, macular degeneration, emphysema, atherosclerosis, and scleroderma.

Apoptosis occurring during normal development is non inflammatory and is involved in induction of immunological tolerance. Although apoptotic cell death can be inflammatory depending on how it is activated and in what cell types (for example, therapeutic agents that ligate Fas are able to induce inflammation), necrotic cell death results in a sustained and powerful inflammatory response mediated by released cell contents and by proinflammatory cytokines released by stimulated phagocytes. Apoptotic cells and vesicles are normally cleared by phagocytes, thus preventing the pro-inflammatory consequences of cell lysis. In this context, it has been shown that apoptotic cells and apoptotic bodies directly fix complement, and that complement can sustain an anti-inflammatory response due to opsonization and enhanced phagocytosis of apoptotic cells.

Inflammation is involved in non specific recruitment of immune cells that can influence innate and adaptive immune responses. Modulating complement activation during apoptosis-based tumor therapy to inhibit phagocytic uptake of apoptotic cells/bodies enhances the inflammatory/innate immune response within the tumor environment. In addition, apoptotic cells can be a source of immunogenic self antigens and uncleared apoptotic bodies can result in autoimmunization. In addition to creating an enhanced immuno-stimulatory environment, modulating complement at a site in which tumor cells have been induced to undergo apoptosis further augments or triggers specific immunity against a tumor to which the host is normally tolerant.

Also disclosed are methods of the invention, wherein the condition treated is a viral infection. The viral infection can be selected from the list of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian immunodeficiency virus, Human Immunodeficiency virus type-1, and Human Immunodeficiency virus type-2.

Also disclosed are methods of the invention, wherein the condition treated is a bacterial infection. Also disclosed are methods of the invention, wherein the bacterial infection can be selected from the list of bacterium consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichlia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods of the invention, wherein the condition treated is a parasitic infection. Also disclosed are methods of the invention, wherein the parasitic infection can be selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

Also disclosed are methods of the invention, wherein the condition treated is a fungal infection. Also disclosed are methods of the invention, wherein the fungal infection can be selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

In the methods of the invention, the subject can be a mammal. For example, the mammal can be a human, nonhuman primate, mouse, rat, pig, dog, cat, monkey, cow, or horse.

### E. METHODS OF USING THE COMPOSITIONS TO ACTIVATE COMPLEMENT

Disclosed herein are modified CD59 molecules wherein the substitution decreases the inhibitory activity of CD59. By decreasing the inhibitory activity of CD59, the disclosed modified CD59 molecules increase complement activity. Thus, for example, are modified CD59 molecules of the invention wherein the modified CD59 molecule decreases the inhibitory activity of CD59 and wherein the modified CD59 molecule comprises a substituted residue selected from the group of residues consisting of 23, 24, 42, and 44. Thus, for example, disclosed herein are modified CD59 molecules of the invention comprising a substitution selected from the group consisting of F23G, D24A, F42A, and H44A.

It is also contemplated herein that the disclosed modified CD59 molecules can comprise more than one substitution. Thus, disclosed herein are mutCD59 molecules comprising a substitution at 1, 2, 3, 4, 5, 6, 7, 8, 9, 10, 11, 12, 13, 14, or 15 amino acid residues, wherein the substituted residues can be selected from the group consisting of N18Q, S20A, S21A, D22A, F23A, F23G, L27A, T29A, L33A, Q34A, Y36A, N37A, K38A, W40A, F42A, K41A, T51A, R53A, L54A, R55A, and N57A. It is understood and herein contemplated that multiple substitutions in the modified CD59 molecules of the invention can have an additive effect on the decrease of the inhibitory activity of CD59 relative to a single substitution. Thus, for example, disclosed herein are modified CD59 molecules of the invention wherein the modified CD59 further comprises a second substitution at residues 18, 20, 21, 22, 23, 24, 27, 29, 37, 42, 44, 48, 51, 53, 54, or 57. Thus, for example, disclosed herein are modified CD59 molecules of the invention comprising alanine substitutions at residues 20 and 23. Also disclosed are modified CD59 molecules of the invention comprising an N18Q substitution and an alanine substitution at 29.

Disclosed are methods of treating cancer comprising administering to a subject with cancer the modified CD59 molecule of the invention, wherein the modified CD59 molecule decreases CD59 inhibition of complement. Also disclosed are methods of the invention, wherein the cancer can be selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumours, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

The complisitions disclosed herein can also be used for the treatment of precancer conditions such as cervical and anal dysplasias, other dysplasias, severe dysplasias, hyperplasias, atypical hyperplasias, and neoplasias. Disclosed are methods of the invention, wherein the condition is a precancer conditions. It is understood that the composition will recognize antigens that are overexpressed on the surface of precancerous cells

In one embodiment of the invention CR2 or like complement targeting moiety can target complement deposited on tumor cells as a result of administered anti-tumor antibodies, or as a result of a normally ineffective humoral immune response.

Thus the present complement activating composition can be administered in conjunction with anti-tumor antibodies. Examples of such anti-tumor antibodies are well known and include anti-PSMA monoclonal antibodies J591, PEQ226.5, and PM2P079.1 (Fracasso, G. et al., (2002) Prostate 53(1): 9-23); anti-Her2 antibody hu4D5 (Gerstner, R. B., et al., (2002) J. Mol. Biol. 321(5): 851-62); anti-disialosyl Gb5 monoclonal antibody 5F3 which can be used as an anti renal cell carcinoma antibody (Ito A. et al., (2001) Glycoconj. J. 18(6): 475-485); anti MAGE monoclonal antibody 57B (Antonescu, C. R. et al., (2002) Hum. Pathol. 33(2): 225-9); anti-cancer monoclonal antibody CLN-Ig (Kubo, O. et al., (2002) Nippon Rinsho. 60(3): 497-503); anti-Dalton's lymphoma associated antigen (DLAA) monoclonal antibody DLAB (Subbiah, K. et al., (2001) Indian J. Exp. Biol. 39(10): 993-7). The present composition can be administered before, concurrent with or after administration of the anti-tumor antibody, so long as the present composition is present at the tumor during the time when the antibody is also present at the tumor.

In the methods of the invention, the subject can be a mammal. For example, the mammal can be a human, nonhuman primate, mouse, rat, pig, dog, cat, monkey, cow, or horse.

1. Nucleic Acids

There are a variety of molecules disclosed herein that are nucleic acid based, including for example the nucleic acids that encode, for example CD59 or mutCD59, or any of the nucleic acids disclosed herein for making CD59 or mutCD59 binding inhibitory ligands and protein mimetics. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example CD59 or mutCD59, or any of the nucleic acids disclosed herein for making CD59 or mutCD59, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of CD59 or mutCD59 or the genomic DNA of CD59 or mutCD59 or they can interact with the polypeptide CD59 or mutCD59. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of CD59 or mutCD59 aptamers, the background protein could be CD59 OR MUTCD59. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

2. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way. The CD59 or mutCD59 nucleic acids, peptides, and related molecules disclosed herein can be used as targets for the combinatorial approaches. Also disclosed are the compositions that are identified through combinatorial techniques or screening techniques in which the compositions disclosed in any of the disclosed sequences or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions are also disclosed. Thus, the products produced using the combinatorial or screening approaches that involve the disclosed compositions, such as, CD59 or mutCD59, are also considered herein disclosed.

It is understood that the disclosed methods for identifying molecules that inhibit the interactions between, for example, CD59 or mutCD59 and C8 or C9 can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23) 12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example an extracellular portion of CD59 or mutCD59 is attached to a DNA binding domain of a transcriptional activation protein, such as Gal 4. By performing the Two-hybrid technique on this type of system, molecules that bind the extracellular portion of CD59 or mutCD59 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interactive processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions are also disclosed. Thus, the products produced using the molecular modeling approaches that involve the disclosed compositions, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

3. Nucleic Acid Delivery

In the methods described above for delivering a mutCD59-encoding nucleic acid to a subject by the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the mutCD59-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing mutCD59 (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the mutCD59-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as CD59 or mutCD59 into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially, a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed CD59 or mutCD59, vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

5. Antibodies 1

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with CD59 or mutCD59 such that CD59 or mutCD59 is inhibited from interacting with C8 or C9 or they are chosen for their ability to target a CD59 or mutCD59 to a site of inflammation. Antibodies that bind the disclosed regions of CD59 or mutCD59 involved in the interaction between CD59 or mutCD59 and C8 or C9 are also disclosed. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 199-1; Marks et al., J. Mol. Biol., 222:5-81, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRS) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.); U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti CD59 or mutCD59 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid/solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing an inflammatory condition, the efficacy of the therapeutic antibody can be assessed in various ways well known to the skilled practitioner. For instance, one of ordinary skill in the art will understand that a composition, such as an antibody, disclosed herein is efficacious in treating or inhibiting an inflammatory condition in a subject by observing that the composition reduces Mac dependent pathology.

The disclosed compositions and methods can also be used for example as tools to isolate and test new drug candidates for a variety of complement related diseases.

7. Compositions with Similar Functions

It is understood that the compositions disclosed herein have certain functions, such as binding the binding groove of CD59 or binding to C8 or C9. Disclosed herein are certain structural requirements for performing the disclosed functions, and it is understood that there are a variety of structures which can perform the same function which are related to the disclosed structures, and that these structures will ultimately achieve the same result, for example stimulation or inhibition MAC formation.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Results

(1) Effect of Mutations on CD59 Function

Previous studies have mapped the binding interface of human CD59 to the vicinity of a hydrophobic groove around W40 on the membrane-distal face of the molecule. Rational mutagenesis studies within this region indicated that W40, R53, L54, and E56 are involved in ligand binding, with D24 being crucial for the structure and accessibility of the binding interface (Yu, J., et al. (1997) Journal of Experimental Medicine 185, 745-753, Bodian, D. L., et al. (1997) Journal of Experimental Medicine 185, 507-516). A systematic mutagenesis screening study was performed together with functional analyses to better define the binding site. Thirty-four alanine substitutions were made in a mutagenesis screen of residues 16 through 57 (except for cysteine residues and existing alanine residues), representing the primary sequence known to contain the binding interface (Yu, J., et al. (1997) Journal of Experimental Medicine 185, 745-753).

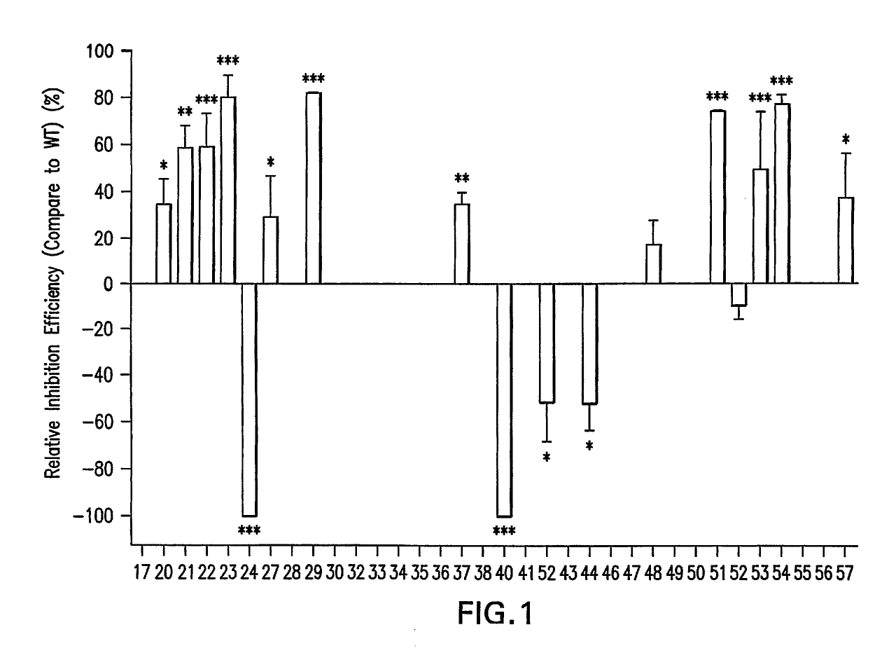

Wild type and mutant proteins were recombinantly expressed on the surface of CHO cells, and cell populations expressing similar levels of protein were isolated by cell sorting by means of an epitope tag. Only 4 of the alanine substitutions significantly reduced the inhibitory activity of CD59 as measured by the complement susceptibility of CHO cells expressing the mutant proteins (FIG. 1). For mutations showing decreased inhibitory activity, expression and folding on the CHO cell surface was verified using a panel of anti-CD59 antibodies that recognize conformational epitopes (Table 3).

TABLE 3
Reactivity of CD59 mutants to anti-CD59 antibodies

| Mutant | Poly | YTH | 1F5 | P282 | MEM43 | HC1 | MEM43.5 |
|---|---|---|---|---|---|---|---|
| WT | 100^{a} | 100 | 100 | 100 | 100 | 100 | 100 |
| 24A | 5.7 ± 1.7 | 5.0 ± 1.8 | 2.8 ± 0.2 | 5.6 ± 0.5 | 3.5 ± 2.5 | 2.2 ± 0.2 | 1.9 ± 0.6 |
| 40A | 4.3 ± 0.1 | 1.0 ± 0.1 | 2.2 ± 1.3 | 3.3 ± 0.1 | 6.2 ± 8.3 | 1.2 ± 0.8 | 0.7 ± 0.4 |
| 42A | 99.7 ± 0.5 | 99.8 ± 0.4 | 99.8 ± 0.3 | 100 ± 0.1 | 99.7 ± 0.4 | 98.4 ± 2.3 | 99.9 ± 0.2 |
| 44A | 82.7 ± 6.4 | 58.7 ± 5.1 | 74.0 ± 3.3 | 95.6 ± 4.9 | 88.8 ± 13.8 | 30.3 ± 10 | 85.9 ± 8.8 |
| 52A | 97.6 ± 1.4 | 99.3 ± 0.1 | 99.0 ± 0.2 | 100 ± 0 | 99.2 ± 0.8 | 90.7 ± 2.9 | 99.2 ± 0.1 |
| 23G | ND^{b} | 99.3 ± 0.1 | 97.5 ± 0.2 | 99.2 ± 0.1 | 98.8 ± 0.1 | 91.3 ± 1.5 | 98.9 ± 0.1 |
| 20A23A | ND | 35.7 ± 1.1 | 83.1 ± 14 | 99.6 ± 0.1 | 98.1 ± 0.2 | 63.1 ± 21 | 97.7 ± 1.0 |
| 51A20-23A | ND | 18.3 ± 1.2 | 64.4 ± 26 | 96.7 ± 0.1 | 92.3 ± 0.4 | 43.5 ± 25 | 98.1 ± 1.5 |
| 5R | 40.7 ± 8.0 | 47.9 ± 11 | 14.8 ± 5.3 | N/D | N/D | 6.3 ± 8.5 | 8.2 ± 3.9 |
| 9R | 100 | 100 | 99.8 | N/D | N/D | 98.7 | 98.4 |

^{a}Percent of relative mean fluorescence by flow cytometry compared to wt CD59
^{b}ND, not determined.

The F42A and H44A substitutions resulted in proteins with significantly reduced inhibitory activity (p<0.05) and antibody binding affinity comparable to wild type CD59, the one exception being HC 1 binding to H44A. D24A and W40A displayed evidence of mis-expression/folding, showing less than 10% reactivity with all antibodies. However, in previous mutational studies, D24R and W40E have been expressed with intact protein topology and with total loss of CD59 activity (Bodian, D. L., et al. (1997) Journal of Experimental Medicine 185, 507-516).

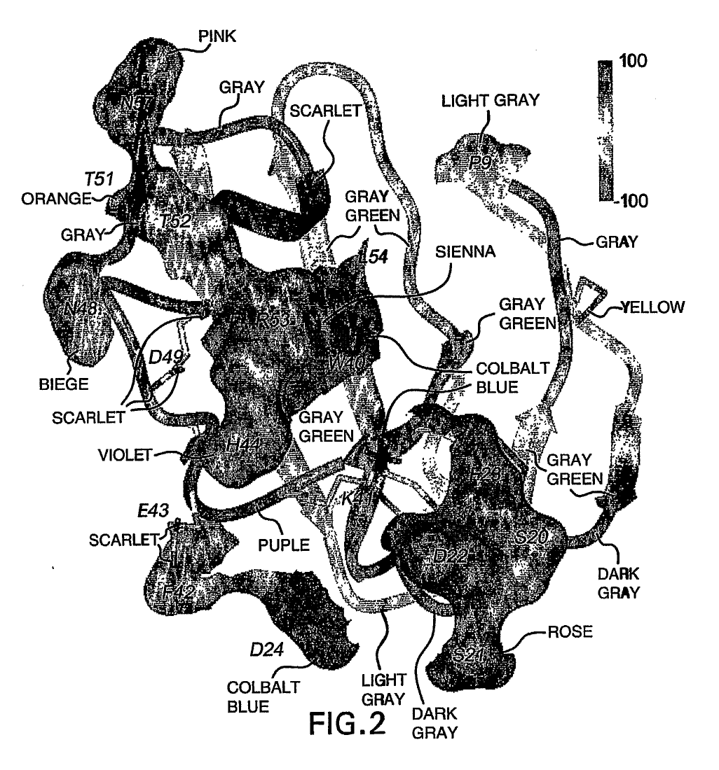

Surprisingly, 11 mutations significantly increased complement inhibitory activity (FIG. 1). Of these mutants, 5 (D22A, F23A, T29A, T51A, L54A) had a marked increase in activity (60%-80%, p<0.001) and 6 mutants (S20A, S21A, L27A, N37A, R53A, N57A) showed a more moderate increase in inhibitory activity (p<0.05 or p<0.01). Interestingly, a number of the mutated residues were internal and localized to the beta sheet (L27A, T29A, N37A). This localization of mutated residues can induce conformational changes of the binding interface or increase protein stability. The solvent exposed mutants that increased activity were localized to two compact locations, the S20-F23 loop and the N48-N57 helix/loop region (FIG. 2).

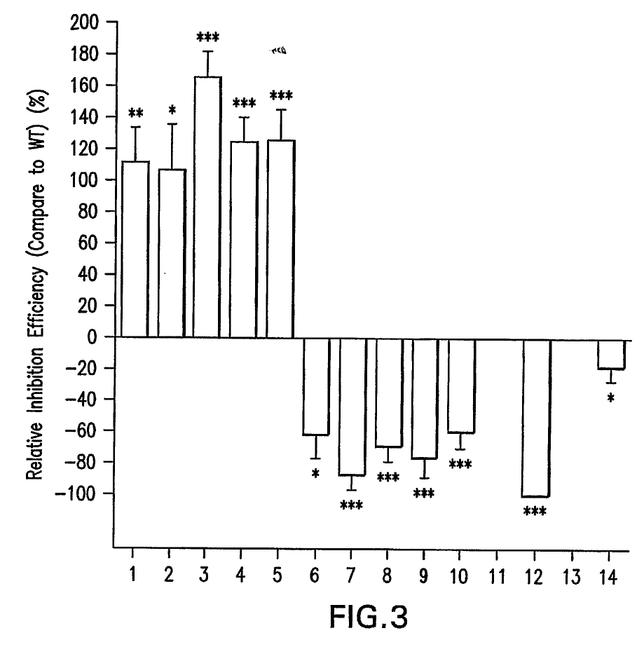

Due to the usefulness of CD59 as a therapeutic agent for preventing tissue injury in some pathological conditions, single residue mutations were combined in an attempt to further increase the complement inhibitory activity of CD59. Six mutant CD59 proteins were prepared that contained alanine substitutions at two different positions (nos. 1-6 in FIG. 3). Five of the “double-mutant” proteins (29A21A, 29A23A, 51A20A, 51A23A and 51A29A) enhanced CD59 inhibitory activity and showed additive activity compared to the single-residue mutations. The most potent inhibitor was the 51A20A mutant, which showed a 165% increase in inhibitory activity compared to the wt protein (p<0.001) (FIG. 3). This double mutation exhibited twice the inhibitory activity of the highest single mutation, T29A. In contrast, one double alanine mutant (20A23A), together with other mutant proteins containing multiple alanine or glycine substitutions with more than one substitution in the S20-F24 loop, displayed significantly decreased inhibitory activity compared to the wt protein (p<0.05 or p<0.001) (FIG. 3). A single 23G substitution also resulted in decreased CD59 activity. An explanation lies in the fact that residues S20, S21, and D22 form a polar loop with almost entirely solvent exposed side chains. The backbone of these residues is partially supported by the bulky phenyl group of F23. Without support of the phenyl group, the loop is more prone to collapse triggered by substitution of the polar residues with more hydrophobic alanine or glycine. Such a local conformational change is evidenced by the partial disruption of YTH53.1, 1F5, and HC1 antibody reactivity to 20A23A and 51A20-23A (Table 3). F23G, on the other hand, shows no disruption in antibody binding. The difference in CD59 activity of the F23A and F23G mutants is attributable to the structural importance of the additional carbon atom in F23A. Since an the absence of N-glycosylation by an N18Q substitution was previously shown to enhance CD59 activity (Yu, J., et al. (1997) Journal of Experimental Medicine 185, 745-753, Akami, T., et al. (1994) Transp. Proc. 26, 1256-1258), a 29A18Q mutant was also prepared, but was found to display reduced activity

(2) Effect of CD59 Mutations on CR2-CD59 Fusion Protein Activity

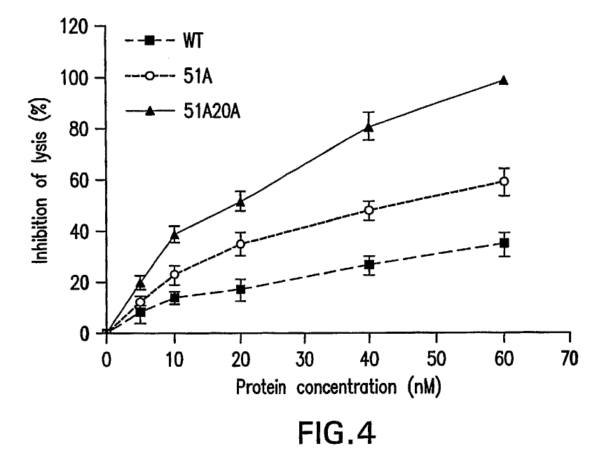

For CD59 to function effectively, it must be bound close to the site of complement activation and MAC formation (Song, H., et al. (2003) J Clin Invest 111(12), 1875-1885, Zhang, H.-F., et al. (1999) J. Clin. Invest. 103, 55-66). Soluble CD59 is only a poor inhibitor of complement (MAC formation), and it was demonstrated that the functional activity of CD59 could be markedly enhanced by targeting CD59 to the site of complement activation (Song, H., et al. (2003) J Clin Invest 111(12), 1875-1885, Zhang, H.-F., et al. (1999) J. Clin. Invest. 103, 55-66). By linking the extracellular region (residues 1-77) of CD59 to a C3 binding region of complement receptor 2 (CR2), it was demonstrated that a significant increase in the ability of CD59 to protect target cells from complement-mediated lysis (Song, H., et al. (2003) J Clin Invest 111(12), 1875-1885). With the idea of developing an improved therapeutic agent by further enhancing the activity of CD59, two soluble CD59 molecules containing mutations that increased activity (when linked via GPI anchor to the cell surface) were linked to a CR2 targeting moiety. CR2-CD59 fusion proteins were prepared containing wild type CD59 (SEQ ID NOs: 9 and 10) or CD59 containing either a T51A (SEQ ID NOs: 11 and 12) mutation or a combined mutation of T51A/S20A (SEQ ID NOs: 13 and 14). The proteins were assayed for their ability to protect sensitized CHO cells from complement-mediated lysis, and both mutant CR2-CD59 proteins displayed significantly enhanced complement inhibitory activity compared to the wt CR2-CD59 protein, with CR2-CD59 (T51A/S20A) displaying an approximate 3-fold increase in activity (FIG. 4).

b) Materials and Methods

(1) Cell Line and DNA

CHO cells were used for CD59 expression using F12K medium (GIBCO, Gaithersburg, Md.) supplemented with 10% FCS (Mediatech, Herndon, Va.). Plasmid pCDNA3/CD59 (Yu, J., et al. (1997) Journal of Experimental Medicine 185, 745-753) was used as the starting DNA for all mutagenesis experiments. The plasmid carries human CD59 cDNA sequence including sequence coding for the addition of a GPI anchor. To facilitate investigation of CD59 expression, an epitope tag consisting of amino acids NANPNANPNA (SEQ ID NO: 3) was inserted immediately behind the first amino acid of the N-terminus of CD59 (Yu, J., et al. (1997) Journal of Experimental Medicine 185, 745-753). The presence of this tag has been shown to have no effect on the function of CD59. pCDNA3 carries a G418 resistance gene and 100 μg/ml G418 (GIBCO) was supplemented for selection and cultivation of transfected CHO cells and populations.

(2) Antibodies, Sera and Reagents

Rabbit anti-CD59 polyclonal antibody was prepared by standard techniques (Harlow, E., and Lane, D. (1988) Antibodies. A laboratory manual, Cold Spring Harbor Laboratory, New York). Anti-CD59 mAbs YTH53.1, 1F5, and anti-tag mAb 2A10 were expressed in our lab. The remaining anti-CD59 mAbs were from Dr. V. Horejsi, Academy of Sciences of the Czech Republic, Prague (p282 and MEM43.5), Dr. S. Meri, University of Helsinki, Finland (HC1) and from BD Biosciences, San Jose, Calif. (MEM43). All FITC conjugated secondary antibodies were from Sigma-Aldrich (St. Louis, Mo., USA). Rabbit anti-CHO cell membrane antiserum was prepared by standard techniques (Harlow, E., and Lane, D. (1988) Antibodies. A laboratory manual, Cold Spring Harbor Laboratory, New York). Normal human serum (NHS) was prepared from blood of healthy volunteers. All other reagents were from Sigma.

(3) Mutagenesis and Transfection

Site-directed mutagenesis was carried out by PCR using the QuikChange® Site-Directed Mutagenesis Kit from Stratagene (La Jolla, Calif.). Two mutagenesis primers spanning the desired mutation site(s) were used for mutagenesis of each mutant. Mutated DNA samples were sequenced to verify the mutations. Wild type and mutant CD59 expression plasmids were transfected into CHO cells using lipofectamine according to manufacture's instruction (GIBCO). Twenty four hr after transfection, G418 was added into F12K medium for selection of stably transfected populations of cells.

(4) Cell Sorting and Antibody Staining

CHO cell populations expressing similar levels of CD59 or mutant CD59 were isolated by cell sorting using a FACS-Vantage™ flow cytometer (Becton Dickinson, San Jose, USA) as described (Yu, J., et al. (1997) Journal of Experimental Medicine 185, 745-753). All cells lines were sorted for 2-4 rounds by means of an antibody to the epitope tag (mAb2A10) in order to acquire population of cells expressing similar levels of CD59. The binding of anti-CD59 antibodies to CD59 and mutant CD59 expressed on CHO cells was performed by flow cytometry using standard methodology as described (Yu, J., et al. (1997) Journal of Experimental Medicine 185, 745-753).

(5) Expression and Purification of CR2-CD59 Fusion Proteins

A cDNA construct was prepared by joining the complement receptor 2 (CR2) sequence encoding the 4 N-terminal SCR units (residues 1-250 of mature protein, Swissprot accession no. P20023) to sequences encoding extracellular region of CD59 as described Song, H., et al. (2003) J Clin Invest 111(12), 1875-1885. The CR2-CD59 construct in expression vector pBMCR2-CD59 Song, H., et al. (2003) J Clin Invest 111(12), 1875-1885, was used as the starting template for preparation of CR2-mutant CD59 fusion proteins. Mutant CR2-CD59 containing plasmids were constructed using the QuikChange® Site-Directed Mutagenesis Kit (see above). The CR2-CD59 proteins were expressed after transient transfection of CHO cells by the lipofectamine method. Wild type protein was expressed in suspension culture of transfected CHO cells and recombinant proteins were purified from CHO cell supernatant by anti-CD59 (mAb YTH53.1) affinity chromatography as described Song, H., et al. (2003) J Clin Invest 111(12), 1875-1885. Purity of eluted proteins was determined (>95%) by 10% SDS-PAGE and by Western blotting.

(6) Complemen-Mediated Cho Cell Lysis Assays

CHO cells at 60-80% confluency were detached with 5 mM EDTA in PBS, washed once with PBS and re-suspended to 10⁶/ml in DMEM. Cells were sensitized with 5% rabbit anti-CHO membrane serum and 10% NHS diluted in DMEM was then added (final volume 400 μl). Following incubation at 37° C. for 60 min, cell viability was determined by adding propidium iodide (PI) (5 μg/ml) and measuring the proportion of PI-stained (dead) cells by flow cytometery. Cells lysed with 0.01% saponin were used as 100% lysis controls and samples with NHS heated at 56° C. for 30 min. were used for background lysis. To test the complement inhibitory function of wt and mutant CR2-CD59 fusion proteins, the proteins were diluted in DMEM and added to NHS prior to addition to sensitized CHO cells. Cell viability was determined by both trypan blue exclusion (both live and dead cells counted) and PI staining. Both methods gave similar results.

c) Discussion

Disclosed herein are two new residues, F42 and H44, which are shown to be involved in the binding interface of human CD59. Mutations of these residues to alanine resulted in proteins with significantly reduced complement inhibitory function, yet intact overall structure. Both residues are in close proximity to the proposed binding interface. Of relevance to this data, glycation of CD59 decreases its function and has been shown to occur in diabetic patients due to hyperglycemia. It has been shown that H44, together with K41 that is adjacent to the functionally important W40, form a preferential glycation motif in human CD59 (Acosta, J., et al. (2000) Proc Natl Acad Sci USA 97(10), 5450-5455, Qin, X., et al. (2004) Diabetes 53(10), 2653-2661, Davies, C. S., et al. (1995) J. Biol. Chem. 270, 19723-19728). The results disclosed herein also indicate that residues 20-22 are involved in the activity of human CD59. Based on the NMR structure of soluble CD59, Fletcher et al. (Fletcher, C. M., et al. (1994) Structure 2(3), 185-199) suggested that the S20-D24 loop is part of the binding interface, a supposition further supported by mutational analysis (Yu, J., et al. (1997) Journal of Experimental Medicine 185, 745-753, Bodian, D. L., et al. (1997) Journal of Experimental Medicine 185, 507-516, Hinchliffe, S. J., and Morgan, B. P. (2000) Biochemistry 39(19), 5831-5837). Though mutations of D24 and F23 reduced the function of human CD59, mutagenesis of residues 20-22 in human CD59 has not previously been reported, and mutations of residues 20-23 in rat CD59 showed no effect on CD59 function (Hinchliffe, S. J., and Morgan, B. P. (2000) Biochemistry 39(19), 5831-5837). However, in this study, S20A, S21A, D22A and F23A significantly improved the inhibitory activity of CD59, indicating their involvement in CD59 binding. Efforts to further increase CD59 activity through synergistic mutations of those residues proved unsuccessful (and resulted in decreased activity compared to wild type CD59) and indicate that excessive localized mutation within this loop causes significant deformations.

Interestingly, three activity-enhancing mutations (L27A, T29A, N37A) are located at the center of the protein in two strands of the beta sheet, and confer increased activity through induced conformational changes. One mechanism for the increased activity is the spatial relationship of these residues with W40. Previous studies have demonstrated the importance of W40 in CD59 activity (Bodian, D. L., et al. (1997) Journal of Experimental Medicine 185, 507-516). This study shows that W40 is almost entirely surrounded by residues (L27/T29 from the beta sheet and R53/L54 from the helix-loop), that when mutated to alanine, cause an increase in inhibitory activity. This may indicate that the mobility/accessibility of W40 is a key factor in determining the activity of CD59.

The comprehensive mutational data presented here indicates a larger C8/C9 binding interface than previously thought. Significantly, the mutation that produced the highest increase in inhibitory activity, 51A20A, involves residues on nearly opposite ends of CD59. Additionally, the mutational data indicates broadening the definition of the binding interface to include F42 and H44. The new model of CD59 provides an exposed face offset by approximately 90 degrees. Altogether, the data indicate a protein interface where CD59 interacts with C8/C9 on multiple discontinuous sites. Of potential relevance to this finding, previous studies on the interaction of C8 with CD59 identified a region of the C8α chain that is critical for C8 binding to human CD59, but the data also indicated that additional portions of the C8 molecule were involved in the interaction (Lockert, D. H., et al. (1995) J. Biol. Chem. 270, 19723-19728).

Mutation of a single residue in rat CD59 (K48) resulted in enhanced activity (Hinchliffe, S. J., and Morgan, B. P. (2000) Biochemistry 39(19), 5831-5837), as did mutational disruption of the cysteine 64-69 disulfide bond in human CD59 (Petranka, J., et al. (1996) Blood Cell. Mol. Dis. 22, 281-295). However, the discovery of the numerous alanine mutations that enhance CD59 activity is surprising and interesting with regard to the evolutionary pressures on CD59. It was shown herein that numerous single-residue mutations in CD59 can dramatically increase its ability to prevent complement-mediated cell lysis.

Demonstrated herein is the capacity to engineer CD59 constructs with increased activity through single-alanine mutations alone. Also shown herein is the negative synergy of multiple highly-localized mutations, with effects possibly mediated through local conformational changes. On the other hand, combined point mutations in different locations preserved the overall conformation and produced synergistic increases in C8/C9 binding affinity. Use of this principle in future mutational studies can provide further increases in CD59 inhibitory activity.

CD59 has other functions beyond complement inhibition and can therefore have other protein interaction interfaces. In that regard, a particularly interesting observation we made is the “back” patch on the CD59 surface (C45, N46, F47, T60, Y61, Y62, L75) with a strong protein interface signal. These residues are good candidates for involvement in the interaction of CD59 with its other identified ligands.

Through an extensive mutagenesis screen, the binding interface of CD59 has been further defined. With the inclusion of D22, F23, F42 and H44, the area of the binding interface is much broader than previously thought. Also identified are small-molecule binding sites directly interfering with the binding interface, which makes the rational design of efficient CD59 inhibitors more feasible. Inhibitors of CD59 function, if appropriately targeted, may be effective at enhancing antibody immunotherapy. Finally, we show that mutations that enhance the activity of GPI-linked membrane CD59 also significantly enhance the activity of soluble CD59 constructs (CR-CD59 fusion proteins), thus establishing that more effective CD59-based therapeutics can be engineered to treat inflammatory conditions.

### 2. Example 2

The disclosed teachings herein describe the synthesis and composition of fusion proteins and immunoconjugates. Provided below are examples of fusion proteins and immunoconjugates that can be made using the teachings herein. It is understood that any of the modified CD59 molecules described can be used and are herein are specifically contemplated as being interchangeable with the CD59 molecules provided.

CR2-(Gly₄Ser)₃-human CD59(F23A)

CR2-(Gly₄Ser)₃-human CD59(T29A)

CR2-(Gly₄Ser)₃-human CD59(T51A)

CR2-(Gly₄Ser)₃-human CD59(L54A)

CR2-(Gly₄Ser)₃-human CD59(S20A)

CR2-(Gly₄Ser)₃-human CD59(S21A)

CR2-(Gly₄Ser)₃-human CD59(D22A>

CR2-(Gly₄Ser)₃-human CD59(L27A)

CR2-(Gly₄Ser)₃-human CD59(N37A)

CR2-(Gly₄Ser)₃-human CD59(D24A)

CR2-(Gly₃Ser)₄-human CD59(F23A)

CR2-(Gly₃Ser)₄-human CD59(T29A)

CR2-(Gly₃Ser)₄-human CD59(T51A)

CR2-(Gly₃Ser)₄-human CD59(L54A)

CR2-(Gly₃Ser)₄-human CD59(S20A)

CR2-(Gly₃Ser)₄-human CD59(S21A)

CR2-(Gly₃Ser)₄-human CD59(D22A)

CR2-(Gly₃Ser)₄-human CD59(L27A)

CR2-(Gly₃Ser)₄-human CD59(N37A)

CR2-(Gly₃Ser)₄-human CD59(D24A)

CR2-VSVFPLE-human CD59(F23A)

CR2-VSVFPLE-human CD59(T29A)

CR2-VSVFPLE-human CD59(T51A)

CR2-VSVFPLE-human CD59(L54A)

CR2-VSVFPLE-human CD59(S20A)

CR2-VSVFPLE-human CD59(S21A)

CR2-VSVFPLE-human CD59(D22A)

CR2-VSVFPLE-human CD59(L27A)

CR2-VSVFPLE-human CD59(N37A)

CR2-VSVFPLE-human CD59(D24A)

CR2-m-Maleimidobenzoyl-N-hydroxysuccinimide ester-human CD59(F23A)

CR2-m-Maleimidobenzoyl-N-hydroxysuccinimide ester-human CD59(T29A)

CR2-m-Maleimidobenzoyl-N-hydroxysuccinimide ester-human CD59(T51A)

CR2-m-Maleimidobenzoyl-N-hydroxysuccinimide ester-human CD59(L54A)

CR2-m-Maleimidobenzoyl-N-hydroxysuccinimide ester-human CD59(S20A)

CR2-m-Maleimidobenzoyl-N-hydroxysuccinimide ester-human CD59(S21A)

CR2-m-Maleimidobenzoyl-N-hydroxysuccinimide ester-human CD59(D22A)

CR2-m-Maleimidobenzoyl-N-hydroxysuccinimide ester-human CD59(L27A)

CR2-m-Maleimidobenzoyl-N-hydroxysuccinimide ester-human CD59(N37A)

CR2-m-Maleimidobenzoyl-N-hydroxysuccinimide ester-human CD59(D24A)

CR2-bismaleimidohexane-human CD59(F23A)

CR2-bismaleimidohexane-human CD59(T29A)

CR2-bismaleimidohexane-human CD59(T51A)

CR2-bismaleimidohexane-human CD59(L54A)

CR2-bismaleimidohexane-human CD59(S20A)

CR2-bismaleimidohexane-human CD59(S21A)

CR2-bismaleimidohexane-human CD59(D22A)

CR2-bismaleimidohexane-human CD59(L27A)

CR2-bismaleimidohexane-human CD59(N37A)

CR2-bismaleimidohexane-human CD59(D24A)

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### G. REFERENCES

1. Arumugam, T. V., Shiels, I. A., Woodruff, T. M., Granger, D. N., and Taylor, S. M. (2004) Shock 21(5), 401-409.
2. Morgan, B. P., and Harris, C. L. (2003)Mol Immunol 40(2-4), 159-170.
3. Sahu, A., and Lambris, J. D. (2000) Immunopharmacology 49(1-2), 133-148.
4. Quigg, R. J. (2002) Trends Mol Med 8(9), 430-436.
5. Lambris, J. D., and Holers, V. M. (eds). (2000) Therapeutic interventions in the complement system, Humana Press, Totowa, N.J.
6. Chen, S., Caragine, T., Cheung, N. K., and Tomlinson, S. (2000) Cancer Res. 60, 3013-3118.
7. Shapiro, A., Kelley, D. R., Oakley, D. M., Catalona, W. J., and Ratliff, T. L. (1984) Cancer Res 44(7), 3051-3054.
8. Gelderman, K. A., Tomlinson, S., Ross, G. D., and Gorter, A. (2004) Trends Immunol 25(3), 158-164.
9. Fishelson, Z., Donin, N., Zell, S., Schultz, S., and Kirschfink, M. (2003) Mol Immunol 40(2-4), 109-123.
10. Murray, K. P., Mathure, S., Kaul, R., Khan, S., Carson, L. F., Twiggs, L. B., Martens, M. G., and Kaul, A. (2000) Gynecol Oncol 76(2), 176-182.
11. Thorsteinsson, L., O'Dowd, G. M., Harrington, P. M., and Johnson, P. M. (1998) APMIS 106 Fishelson, Z., et al. (2003) Mol Immunol 40(2-4), 109-123, 869-878.
12. Xu, C., Jung, M., Burkhardt, M., Stephan, C., Schnorr, D., Loening, S., Jung, K., Dietel, M., and Kristiansen, G. (2005) Prostate 62(3), 224-232.
13. Kieffer, B., Driscoll, P. C., Campbell, I. D., Willis, A. C., van der Merwe, P. A., and Davis, S. J. (1994) Biochemistry 33(15), 4471-4482.
14. Fletcher, C. M., Harrison, R. A., Lachmann, P. J., and Neuhaus, D. (1994) Structure 2(3), 185-199.
15. Yu, J., Abagyan, R. A., Dong, S., Gilbert, A., Nussenzweig, V., and Tomlinson, S. (1997) Journal of Experimental Medicine 185, 745-753.
16. Bodian, D. L., Davies, S. J., Morgan, B. P., and Rushmere, N. K. (1997) Journal of Experimental Medicine 185, 507-516.
17. Petranka, J., Zhao, J., Norris, J., Tweedy, N. B., Ware, R. E., Sims, P. J., and Rosse, W. F. (1996) Blood Cell. Mol. Dis. 22, 281-295.
18. Hinchliffe, S. J., and Morgan, B. P. (2000) Biochemistry 39(19), 5831-5837.
19. Zhang, H.-F., Yu, J., Chen, S., Morgan, B. P., Abagyan, R., and Tomlinson, S. (1999) J. Biol. Chem. 274, 10969-10974.
20. Zhao, X. J., Zhao, J., Zhou, Q., and Sims, P. J. (1998) J. Biol. Chem. 273, 10665-10671
21. Huesler, T., Lockert, D. H., Kaufman, K. M., Sodetz, J. M., and Sims, P. J. (1995) J. Biol. Chem. 270, 3483-3486.
22. Yu, J., Dong, S., Rushmere, N. K., Morgan, B. P., Abagyan, R., and Tomlinson, S. (1997) Biochem. 36, 9423-9428.
23. Harlow, E., and Lane, D. (1988) Antibodies. A laboratory manual, Cold Spring Harbor Laboratory, New York.
24. Song, H., He, C., Knaak, C., Guthridge, J. M., Holers, V. M., and Tomlinson, S. (2003) J Clin Invest 111(12), 1875-1885.
25. Fletcher, C. M., Harrison, R. A., Lachmann, P. J., and Neuhaus, D. (1993) Protein Sci 2(12), 2015-2027.
26. Abagyan, R., and Totrov, M. (1994) J Mol Biol 235(3), 983-1002.
27. Nemethy, G., Gibson, K. D., Palmer, K. A., Yoon, C. N., Paterlini, G., Zagari, A., Rumsey, S., and Scheraga, H. A. (1992) J Phys Chem 96(15), 6472-6484
28. Totrov, M. (2004) J Comput Chem 25(4), 609-619.
29. Akami, T., Arakawa, K., Okamoto, M., Fujiwara, I., Nakai, I., Mitsuo, M., Sawada, R., Naruto, M., and Oka, T. (1994) Transp. Proc. 26, 1256-1258.
30. Femandez-Recio, J., Totrov, M., Skorodumov, C., and Abagyan, R. (2005) Proteins 58(1), 134-143.
31. Zhang, H.-F., Yu, J., Bajwa, E., Morrison, S. L., and Tomlinson, S. (1999) J. Clin. Invest. 103, 55-66.
32. Acosta, J., Hettinga, J., Fluckiger, R., Krumrei, N., Goldfine, A., Angarita, L., and Halperin, J. (2000) Proc Natl Acad Sci U S A 97(10), 5450-5455.
33. Qin, X., Goldfine, A., Krumrei, N., Grubissich, L., Acosta, J., Chorev, M., Hays, A. P., and Halperin, J. A. (2004) Diabetes 53(10), 2653-2661.
34. Davies, C. S., Harris, C. L., and Morgan, B. P. (2005) Immunology 114(2), 280-286
35. Lockert, D. H., Kaufman, K. M., Chang, C.-P., Huesler, T., Sodetz, J. M., and Sims, P. J. (1995)J. Biol. Chem. 270, 19723-19728.
36. Hahn, W. C., Menu, E., Bothwell, A. L., Sims, P. J., and Bierer, B. E. (1992) Science 256, 1805-1807.
37. Husler et al., Biochemistry. 1996 Mar. 12; 35(10):3263-9
38. Tomlinson et al., J Immunol. 1994 Feb. 15; 152(4):1927-34.
39. Chang et al., J Biol. Chem. 1994 Oct. 21; 269(42):26424-30.
40. Davies, A., Simmons, D. L., Hale, G., Harrison, R. A., Tighe, H., Lachmann, P. J. and Waldmann, H (1989) J. Exp. Med. 170 (3), 637-654.

1. Disclosed herein are modified CD59 molecules wherein the molecule has a first amino acid substitution between residues 16 and 57, wherein the substitution modulates the inhibitory activity of CD59, wherein the substitution does not change a cysteine, and wherein the substitution is not at residue 40.

2. The CD59 molecule of claim 1, wherein the CD59 molecule is a first component of a fusion protein.

3. The fusion protein of claim 2, wherein the fusion protein further comprises CR2.

4. The CD59 molecule of claim 1, wherein the substitution increases the inhibitory activity of CD59.

5-63. (canceled)


SEQUENCE LISTING (14 sequences)

SEQ ID NO: 1 - DNA, 578 bp, Artificial Sequence
  Description of Artificial Sequence note= syntheticconstruct
ctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagca

SEQ ID NO: 2 - protein, 128 aa, Artificial Sequence
  Description of Artificial Sequence note= synthetic construct
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 3 - protein, 10 aa, Artificial Sequence
  Description of Artificial Sequence note= syntheticconstruct
NANPNANPNA

SEQ ID NO: 4 - DNA, 2397 bp, Artificial Sequence
  Description of Artificial Sequence note= synthetic construct
ttttttttttcatcctactttgttttattgggcgttgattgttacaggtcccagcctgtagacatcttttactccaatttcctgaatagatagctttattccttcaaggtaatatagtgcggtggcttctggctgagatgtttgctgttgttttcttcatcttgtctttgatgacttgtcagcctggggtaactgcacaggagaaggtgaaccagagagtaagacgggcagctacacccgcagcagttacctgccagctgagcaactggtcagagtggacagattgctttccgtgccaggacaaaaagtaccgacaccggagcctcttgcagccaaacaagtttgggggaaccatctgcagtggtgacatctgggatcaagccagctgctccagttctacaacttgtgtaaggcaagcacagtgtggacaggatttccagtgtaaggagacaggtcgctgcctgaaacgccaccttgtgtgtaatggagaccaggactgccttgatggctctgatgaggacgactgtgaagatgtcagggccattgacgaagactgcagccagtatgaaccaattccaggatcacagaaggcagccttggggtacaatatcctgacccaggaagatgctcagagtgtgtacgatgccagttattatgggggccagtgtgagacggtatacaatggggaatggagggagcttcgatatgactccacctgtgaacgtctctactatggagatgatgagaaatactttcggaaaccctacaactttctgaagtaccactttgaagccctggcagatactggaatctcctcagagttttatgataatgcaaatgaccttctttccaaagttaaaaaagacaagtctgactcatttggagtgaccatcggcataggcccagccggcagccctttattggtgggtgtaggtgtatcccactcacaagacacttcattcttgaacgaattaaacaagtataatgagaagaaattcattttcacaagaatcttcacaaaggtgcagactgcacattttaagatgaggaaggatgacattatgctggatgaaggaatgctgcagtcattaatggagcttccagatcagtacaattatggcatgtatgccaagttcatcaatgactatggcacccattacatcacatctggatccatgggtggcatttatgaatatatcctggtgattgacaaagcaaaaatggaatcccttggtattaccagcagagatatcacgacatgttttggaggctccttgggcattcaatatgaagacaaaataaatgttggtggaggtttatcaggagaccattgtaaaaaatttggaggtggcaaaactgaaagggccaggaaggccatggctgtggaagacattatttctcgggtgcgaggtggcagttctggctggagcggtggcttggcacagaacaggagcaccattacataccgttcctgggggaggtcattaaagtataatcctgttgttatcgattttgagatgcagcctatccacgaggtgctgcggcacacaagcctggggcctctggaggccaagcgccagaacctgcgccgcgccttggaccagtatctgatggaattcaatgcctgccgatgtgggccttgcttcaacaatggggtgcccatcctcgagggcaccagctgcaggtgccagtgccgcctgggtagcttgggtgctgcctgtgagcaaacacagacagaaggagccaaagcagatgggagctggagttgctggagctcctggtctgtatgcagagcaggcatccaggaaaggagaagagagtgtgacaatccagcacctcagaatggaggggcctcgtgtccagggcggaaagtacagacgcaggcttgctgagggcctctggacacaggctggaccagatgctgtggatgtcgacccctgcactgactattggataaagacttctttcaactaagagaagatgcaaatcagcacacttttttctttgttctgccagcttccaggcctaagactaggttttgctgtctacagccaactattctattagttacaaaactcaatcattttattcagcaactggatgttgactgttaactagaagctctgtcctacttacagcactttggatcatcaaaaaaataaagtaaaatagaaaactgagaaaactcaatccatgaccagggagaacttacaggatgttagagacaaaacaagcagacacctgaaacaatcaacgcccaataaaacaaagtaggatgaaaattctcttagttctttgataacaatttgttcactcatagaaacattattaattggtagggtaagcagacactctgaaacaatgagaaaaatactaaaaattgacttgagttatttc

SEQ ID NO: 5 - protein, 584 aa, Artificial Sequence
  Description of Artificial Sequence note= syntheticconstruct
MFAVVFFILSLMTCQPGVTAQEKVNQRVRRAATPAAVTCQLSNWSEWTDCFPCQDKKYRHRSLLQPNKFGGTICSGDIWDQASCSSSTTCVRQAQCGQDFQCKETGRCLKRHLVCNGDQDCLDGSDEDDCEDVRAIDEDCSQYEPIPGSQKAALGYNILTQEDAQSVYDASYYGGQCETVYNGEWRELRYDSTCERLYYGDDEKYFRKPYNFLKYHFEALADTGISSEFYDNANDLLSKVKKDKSDSFGVTIGIGPAGSPLLVGVGVSHSQDTSFLNELNKYNEKKFIFTRIFTKVQTAHFKMRKDDIMLDEGMLQSLMELPDQYNYGMYAKFINDYGTHYITSGSMGGIYEYILVIDKAKMESLGITSRDITTCFGGSLGIQYEDKINVGGGLSGDHCKKFGGGKTERARKAMAVEDIISRVRGGSSGWSGGLAQNRSTITYRSWGRSLKYNPVVIDFEMQPIHEVLRHTSLGPLEAKRQNLRRALDQYLMEFNACRCGPCFNNGVPILEGTSCRCQCRLGSLGAACEQTQTEGAKADGSWSCWSSWSVCRAGIQERRRECDNPAPQNGGASCPGRKVQTQAC

SEQ ID NO: 6 - DNA, 2094 bp, Artificial Sequence
  Description of Artificial Sequence note= syntheticconstruct
gcttgttccctgtcctctggccctttgcaaataaatgccttaccagacctgccctgccaccccactcgcagccacccagcaagagcagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagatcctacccctgaagataatcttagctgccaagtaaatagcaacatgcttcatgaaaatcctaccaacctctgaagtctcttctctcttaggtctataattttttttttaatttttcttccttaaactcctgtgatgtttccattttttgttccctaatgagaagtcaacagtgaaatacgccagaactgctttatcccacggaaaatgccaatctcttctaaaaaaaaacaaaattaaactaaaaaaaaaaaaaaaaaaaaaaaaaaaaaa

SEQ ID NO: 7 - protein, 559 aa, Artificial Sequence
  Description of Artificial Sequence note= synthetic construct
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 8 - protein, 128 aa, Artificial Sequence
  Description of Artificial Sequence note= syntheticconstruct
MGVQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 9 - DNA, 1002 bp, Artificial Sequence
  Description of Artificial Sequence note= synthetic construct
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgttcaggaggaggaggttccctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaat

SEQ ID NO: 10 - protein, 334 aa, Artificial Sequence [sequence omitted]

SEQ ID NO: 11 - DNA, 999 bp, Artificial Sequence
  Description of Artificial Sequence note= synthetic construct
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgttcaggaggaggaggttccctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaaggacctgtgtaactttaacgaacagcttgaaaat

SEQ ID NO: 12 - protein, 334 aa, Artificial Sequence
  Description of Artificial Sequence note= syntheticconstruct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCSGGGGSLQCYNCPNPTADCKTAVNCSSDFDACLITKAGLQVYNKCWKFEHCNFNDVATRLRENELTYYCCKKDLCNFNEQLEN

SEQ ID NO: 13 - DNA, 996 bp, Artificial Sequence
  Description of Artificial Sequence note= synthetic construct
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgttcaggaggaggaggttccctgcagtgctacaactgtcctaacccaactgctgactgcaaaacagccgtcaattgttcatctgattttgatgcgtgtctcattaccaaagctgggttacaagtgaacaagtgttggaagtttgagcattgcaatttcaacgacgtcacaacccgcttgagggaaaatgagctaacgtactactgctgcaaggacctgtgtaactttaacgaacagcttgaaaat

SEQ ID NO: 14 - protein, 334 aa, Artificial Sequence
  Description of Artificial Sequence note= synthetic construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCSGGGGSLQCYNCPNPTADCKTAVNCSADFDACLITKAGLQVYNKCWKFEHCNFNDVATRLRENELTYYCCKKDLCNFNEQLEN